(19) United States
(12) Patent Application Publication
(10) Pub. No.: US 2023/0129737 A1
(43) Pub. Date: Apr. 27, 2023

(54) Electromagnetic Method and Apparatus

(71) Applicant: Emblation Limited, Inglewood Alloa (GB)

(72) Inventors: Gary Beale, Inglewood Alloa (GB); Eamon McErlean, Inglewood Alloa (GB)

(73) Assignee: Emblation Limited, Inglewood Alloa (GB)

(21) Appl. No.: 17/912,465

(22) PCT Filed: Mar. 18, 2021
(86) PCT No.: PCT/EP2021/057026

§ 371 Date: Sep. 16, 2022

(30) Foreign Application Priority Data
Mar. 18, 2020 (GB) 2003929.3

(51) Int. Cl. A61L 9/18

(52) U.S. Cl. CPC A61L 9/18; A61L 2209/14

(57) ABSTRACT

Apparatus for electromagnetic treatment of at least one pathogen in air or other gas, comprises a waveguide structure including a treatment region within the waveguide structure, wherein the treatment region is configured to receive the air or other gas that contains the at least one pathogen, means for providing electromagnetic radiation to the treatment region, and at least one structure permeable to the air or other gas and that is configured to at least partially confine the electromagnetic radiation, wherein the at least one permeable structure forms at least one boundary of the treatment region.

### FIELD

The present invention relates generally to an apparatus and method for electromagnetic treatment of pathogens in air or other gas.

### BACKGROUND

A viral particle encloses genetic material, either DNA or RNA coated with a protein capsid. These particles as a property of their size and mechanical characteristics possess distinct resonant vibration frequencies. In particular larger viral particles for example influenza and coronaviruses have dimensions in the order of 100s of nanometres and consist of an envelope that comprises negative and positive sensed single stranded RNA segment respectively, see references [3, 4, 5]. The virion structure of these viruses are spherical and at these dimensions these structures possess acoustic resonances in the GHz frequency range. This property has been reported in the literature, see references [1, 2, 6]. Similarly, acoustic resonance properties of non-spherical particles such as rod-shaped viruses has also been elaborated in reference [7].

In many environments air is re-circulated e.g. aeroplanes, hotels etc., and to remove very small particles HEPA filtration is used. HEPA filters must usually remove 99.97% of particles that have a size greater than or equal to 0.3 μm, see reference [8]. As influenza and coronaviruses are dimensionally smaller these filters rely upon the phenomenon of diffusion to enhance the filtration range. Another option would be to employ even finer filter elements, however this places a greater restriction on airflow and a burden on the systems. One of the main limitations of filtration is the requirement to routinely replace the filter membranes as they fill up over time and reduce airflow efficiency.

The launching of circularly polarised waves in a waveguide (e g. a horn antenna) is described in [9].

Waveguides are very widely used for a variety of microwave techniques. Waveguides are often hermetically sealed with barriers/diaphragms or other features to prevent moisture ingress that can attenuate signals, or sealed to prevent corrosion. In some cases, they are filled with an inert gas such as nitrogen

### SUMMARY

Certain aspects of the invention may provide various beneficial methods and delivery modes relating to delivery of microwave energy to eliminate or deactivate viral pathogens in continual flow air systems. Certain aspects relate to destruction of airborne pathogens.

In a first aspect there is provided air permeable electromagnetic boundary(ies) to permit airflow within guided wave structure(s) wherein application of electromagnetic energy is controlled to eliminate or deactivate viral pathogens by means of acoustic resonant destruction at microwave frequencies.

Applied energy may be in the form of a continuous oscillating electromagnetic wave (CW) at a fixed frequency or modulated (variable) frequency. The frequency may range from 5 GHz to 100 GHz but preferentially could be in the microwave range from 7 GHz to 10 GHz to couple to viral particles.

Pulse regimes may include amplitude control of signal energy (AM pulsing) and pulse width modulation control (PWM) and on/off keying (OOK)

Modulation schemes include pulse modulation rate (1-10 kHz) or frequency modulation rate (1-100 kHz).

A continuous dose may be a fixed level of energy or a modulated level of energy during energy delivery. This continuous energy delivery could be pulsed modulated, for example one or five or fifty times a second or with any other suitable frequency during the ongoing energy delivery. Preferentially continuous frequency energy delivery could be pulsed modulated, for example at one thousand times per second (1 KHz) The energy may be applied continuously or as a series of pulses with a time gap between pulses of, for example. 1 s to 60 s may be applied.

Microwave guided wave structures can be of rectangular or cylindrical or other cross-section such as elliptical, curved or square and can be formed into rigid or flexible waveguides.

The or a microwave guided wave structure may operate with any physically possible TE or TM modes above the cut-off frequency. In some cases a mode such as TE10 may possess a peak electric field in the middle of the waveguide (e.g. rectangular) In this case the airflow through a continuous waveguide may become laminar where low velocity airflow may flow close to the sidewalls where the E field is a minimum. It may be preferential to introduce some baffle or finned features inside the structure to perturb this airflow to promote turbulent mixing to ensure more uniform exposure of the air or other gas to the electromagnetic field supported by the propagating mode.

The turbulence inducing features could be metallic and form part of the waveguide walls or be added to contact the walls, or alternatively these features could be low-loss, dielectric insulated materials placed inside the waveguide structure.

The air permeable electromagnetic boundary could be a fine circular or rectangular conductive mesh with hole dimensions having cut-off frequency much higher than the operating frequency, for example by any suitable factors, for instance 2×, 5× or 10×.

The air permeable electromagnetic boundary may be placed on the side wall or broad wall of a rectangular waveguide It may also fully form the part of or the entire waveguide which may, for example, have a rectangular circular, square or elliptical cross-section.

The waveguide may have transition elements that introduce energy from coaxial feeds into the waveguide structure. These transition elements may be introduced from a side feed (e.g. parallel to the waveguide end wall). This could permit an air permeable electromagnetic boundary being used on the end wall of the waveguide. This would preferentially allow a continuous airflow to enter a sealed, for example electromagnetically sealed, waveguide structure via a first transition element, for example a first air permeable electromagnetic boundary, and then to exit via a second transition element, for example a second air permeable electromagnetic boundary.

In a first mode of operation the microwave energy may be delivered into the waveguide as a propagating electromagnetic wave, transported via the structure for a given length and then extracted from the waveguide into a matched absorptive impedance.

In a second mode of operation a resonant cavity may be formed by air permeable electromagnetic boundaries inside the waveguide or to be a portion of the waveguide. In this case the cavity would accept energy at a desired frequency and store this energy within a microwave electromagnetic cavity mode. This has the advantage of requiring significantly less energy to create the high strength electromagnetic field inside the cavity by taking advantage of the resonant properties. This technique has the constraint that the operating frequency can only exist within the bandwidth of the resonant cavity, which can be highly selective and sensitive to temperature and mechanical shock. In addition theory [2] suggests that even low levels of microwave energy can facilitate acoustic destruction of the viral particles without the requirement to employ high-Q resonant cavities

In a further aspect, which may be provided independently, there is provided an electromagnetic air, or other gas, treatment method that utilises a microwave generator to apply microwave energy to air, or other gas, to eliminate, e.g. airborne, pathogens

The method may comprise delivering energy at specific frequency ranges intended to promote continuous acoustic resonant destruction of viral particles suspended and transported in airflow

In a further aspect, which may be provided independently, there is provided an electromagnetic air or other gas treatment system that comprises an energy generator, a waveguide which provides a transmission path for electromagnetic energy from the generator system to an absorbing load, air blowing fan assembly to circulate air and a porous permeable membrane that permits air to enter and exit the waveguide structure but contains the electromagnetic radiation.

In a further aspect, which may be provided independently, there is provided means to couple microwave energy into continuous conduit airflow. This may provide efficient coupling of a microwave photonic oscillation to an acoustic oscillation at the same frequency. The use of a single frequency or plurality of such single frequencies or continuous or discrete sweeping of such frequencies may be used.

The methods and apparatus according to one or more aspects or embodiments may be used to improve filtering of air conditioning equipment as either a primary design function, additional filter stage or retrofitted improvement.

The methods and apparatus according to one or more aspects or embodiments may be used to eliminate or deactivate viral pathogens with microwave energy within a system component not necessarily including filtration and which could potentially be scaled for higher airflows.

In a further aspect, which may be provided independently, there is provided an apparatus for electromagnetic treatment of at least one pathogen in air or other gas. The apparatus may comprise a treatment region configured to receive the air or other gas that contains the at least one pathogen. The apparatus may comprise means for providing electromagnetic radiation. The apparatus may comprise means for providing electromagnetic radiation to the treatment region. The apparatus may comprise at least one structure permeable to the air or other gas and that is configured to at least partially guide and/or confine the electromagnetic radiation

The at least one pathogen may be suspended in the air or other gas.

The at least one permeable structure may be configured to guide the electromagnetic radiation to and/or in the treatment region and/or to at least partially confine the electromagnetic radiation to the treatment region

The at least one permeable structure may provide at least one air permeable electromagnetic boundary

The at least one permeable structure may form at least one boundary of the treatment region.

The treatment region may comprise a cavity

The apparatus may further comprise a waveguide structure and the treatment region may be at least partially, optionally wholly, within the waveguide structure.

The treatment region may comprise a cavity in the waveguide structure.

The at least one permeable structure may be arranged to form the waveguide structure and/or to provide at least one wall of the waveguide structure.

The waveguide structure and/or means for providing electromagnetic radiation means may be configured to provide cavity mode radiation in the treatment region in operation. The electromagnetic radiation may comprise cavity mode radiation. The permeable electromagnetic boundary structures may form the ends of a resonant cavity.

The waveguide structure and/or means for providing electromagnetic radiation means may be configured to provide propagating electromagnetic radiation in the treatment region in operation. The electromagnetic radiation may comprise propagating radiation.

The length of the waveguide cavity may be independent of the mode for propagating mode operation, and dependent on the mode, or the mode may be dependent on the length, for cavity mode operation

The waveguide structure and/or means for providing electromagnetic radiation means may be configured to provide a desired electromagnetic mode in the treatment region, optionally at least one desired TE mode or TM mode.

The waveguide structure may have a desired cross-sectional shape (for example such as to provide desired properties of the electromagnetic radiation), optionally a rectangular, circular, square or elliptical cross-section.

The apparatus may further comprise at least one absorber configured to absorb the electromagnetic radiation

The at least one absorber may comprise at least one matched absorptive impedance

The permeable structure may comprise a substantially gas-impermeable structure that includes at least one opening, preferably a plurality of openings, that allow passage of the air or other gas through the structure.

The or each opening may have dimensions that provide a cut-off frequency higher than a desired frequency of the electromagnetic radiation in the operating region.

The at least one permeable structure may comprise at least one of:
a metal structure;
a mesh structure, a perforated sheet, netting, and/or a filter,
a further waveguide.

The or each permeable structure and/or at least one interior surface of the waveguide structure may include a water-resistant or other protective outer layer, for example a plating or coating, to protect against the effects of atmospheric moisture or other substances that may be present in the air or other gas.

The mesh structure may comprise a circular or rectangular or square mesh.

The at least one permeable structure may comprise at least one curved surface and/or at least one flat surface. The or each surface may be perforated.

The at least one permeable structure may have a desired cross-sectional shape (for example such as to provide desired properties of the electromagnetic radiation), optionally a rectangular, circular, square or elliptical cross-section.

The treatment of the at least one pathogen may comprise at least partially destroying and/or deactivating the at least one pathogen.

The treatment of the at least one pathogen may comprise at least partially inactivating the at least one pathogen. For example, the treatment may yield or render the at least one pathogen unable to replicate infect, and/or colonise a host cell.

The treatment may at least partially, fully and/or substantially decontaminate a volume of air or other gas of at least one pathogen.

The treatment may (at least partially) sterilise a volume of air or other gas

The treatment of the at least one pathogen may comprise rendering the at least one pathogen less harmful and/or less infectious to humans and/or to other living subjects. The treatment of the at least one pathogen may comprise reducing the harm and/or infectiousness of the at least one pathogen.

The electromagnetic radiation may be such as to provide oscillation, optionally acoustic resonance, of the at least one pathogen if present in air or gas in the treatment region.

The oscillation and/or acoustic resonance may be such as to at least partially destroy and/or alter physical structure of and/or at least partially deactivate the at least one pathogen.

The at least one pathogen may comprise at least one virus particle

The at least one pathogen may comprise one or more viral respiratory pathogens

The at least one pathogen may comprise at least one virus particle of the Family Orthomyxoviridae and/or Coronaviridae particles.

The at least one pathogen may comprises at least one virus particle of the Genra Influenzavirus (i.e. Influenza or flu′) and/or Coronavirus.

The at least one pathogen may comprises at least one influenza virus particle.

The pathogen may comprise at least one virus particle classified as any of an Influenza virus A. Influenza virus B, Influenza virus C or Influenza virus D particle

The at least one pathogen may comprise at least one virus particle classified as any of an Avian ‘flu’ (A/H5N1 subtype), a Canine ‘flu’ (Influenza virus), an Equine ‘flu’ (Influenza virus) or a Swine ‘flu’ (A/H1N1 subtype) particles.

The at least one pathogen may comprises at least one virus particle of the Genera Coronavirus.

The at least one pathogen may comprise at least one Coronavirus particle.

The at least one pathogen may comprise at least one virus particle classified as belonging to any of the following Genera: Alpha-, Beta-. Gamma-, and Deltacoronavirus.

The at least one pathogen may comprise at least one virus particle classified as any of the following:

### (i) the SARS Coronavirus; or

### (ii) the MERS Coronavirus: or

(iii) SARS-CoV-2 (aka COVID-19)

The at least one pathogen may comprise at least one SARS-CoV-2 virus particle.

The at least one pathogen may have a non-spherical structure and/or have a non-spherical distribution of electrical charge.

The apparatus may further comprise at least one polariser

The at least one polariser may be arranged such that electromagnetic radiation in at least part of the treatment region and/or in at least part of a cavity of the waveguide structure is at least partially polarised.

The at least one polariser may comprise at least one circular polariser and/or the electromagnetic radiation is circularly polarised

The polariser, or at least one of the polarisers may be arranged such that electromagnetic radiation reflected back from the permeable electromagnetic boundary structure, or one of the permeable electromagnetic boundary structures, passes through the polariser.

The means for providing electromagnetic radiation may comprise a feed structure for feeding electromagnetic energy into a cavity of the waveguide structure The means for providing electromagnetic radiation may comprise a generator or other source of oscillating signal(s) at desired frequency(ies) for example connectable or connected to the feed structure

The polariser may be located between the feed structure and the permeable electromagnetic boundary structure, or one of the permeable electromagnetic boundary structures, and/or the polariser may be located between the feed structure and at least one input arranged to provide the air or other gas to the treatment region.

The polariser(s) and waveguide structure may be configured such that the electromagnetic radiation provides an electric and/or magnetic field that rotates over time with respect to a longitudinal axis of the waveguide structure.

The at least one polariser may comprise at first polariser and a second polariser each towards a respective end of the treatment region and/or waveguide structure and the first polariser may have a desired alignment with respect to the second polariser.

The apparatus may further comprise at least one tuning or perturbation structure, for example that is configured to affect properties of electromagnetic radiation in a cavity of the waveguide structure

The at least one tuning or perturbation structure may comprise at least one conductive or dielectric structure and/or may be configured to provide a capacitance and/or inductance.

The at least one tuning or perturbation structure may be configured to provide a propagating electromagnetic mode at a selected frequency in the waveguide structure.

The permeable electromagnetic boundary structure or at least one of the permeable electromagnetic boundary structures may be configured to reflect at least some of the electromagnetic radiation

The apparatus may comprise a plurality of waveguide structures each including a respective treatment region, wherein the means for means for providing electromagnetic radiation is configured to provide electromagnetic radiation to each of the treatment regions and/or waveguide structures.

The apparatus may further comprise at least one conduit for the air or other gas to pass between the waveguide structures

The plurality of waveguide structures may be provided in a stacked arrangement and/or the plurality of waveguide structures may comprise a plurality of rectangular waveguides.

The apparatus may comprise at least one coaxial connector, or other electromagnetic connector, between adjacent waveguide structures of the plurality of waveguide structures, optionally wherein the at least one coaxial connector, or other electromagnetic connector, passes through an electromagnetically impermeable wall between the adjacent waveguide structures.

The apparatus may further comprise at least one input arranged to provide the air or other gas to the treatment region, and/or at least one output arranged for passage of the air or other gas from the treatment region.

The at least one input may be arranged to provide the air or gas from the at least one input through the at least one permeable structure to the treatment region. The at least one output may be arranged so that the air or other gas passes from the treatment region through the at least one permeable structure to the at least one output.

The waveguide structure may comprise a waveguide including at least part of the treatment region. The at least one input may comprise a further waveguide that functions as the, or one of the, permeable electromagnetic boundary structures.

The further waveguide may have a diameter and/or width and/or height that is less than an operating or cut-off wavelength of the waveguide, and/or the further waveguide may be configured to provide an electromagnetic choke and/or filter effect

The at least one output may be such as to not include a permeable electromagnetic boundary electromagnetic boundary structure and/or may comprise a single open aperture that is configured so as to function as a magnetic boundary, optionally an H-wall boundary

The at least one input may be provided in an end face or a side wall of the waveguide structure, and/or the at least one output may be provided in an end face or a side wall of the waveguide structure.

The input and/or the output may comprise at least one conduit, and the, or at least one of the, permeable electromagnetic boundary structures may be provided in, or at an end of, said at least one conduit.

The apparatus may further comprise means for driving flow of the air or gas to and/or from the treatment region

The driving means may be arranged to drive the air or gas through the at least one permeable structure to the treatment region.

The driving means may comprise at least one pump and/or fan.

The apparatus may further comprise at least one flow control structure configured to control flow of the air or other gas.

The at least one flow control structure may be configured to control flow of the air or other gas in the treatment region

The at least one flow control structure may be configured to provide turbulent flow and/or mixing and/or non-laminar flow of the air or other gas.

The at least one flow control structure may comprise at least one fin and/or baffle.

The at least one flow control structure may comprise or be formed of a dielectric material. The at least one flow control structure may comprise or be formed of metal. The at least one flow control structure may form part of the at least one permeable structure and/or at least one wall of the waveguide structure.

The at least one flow control structure may comprise at least one fan or vent, optionally configured to provide a secondary flow or air or other gas.

The at least one flow control structure may comprise a tubing or guiding structure or other conduit that has a desired form, for example a coiled, curved or otherwise shaped structure that is configured to receive the flow of air or other gas and that passes through the treatment region The tubing or guiding structure or other conduit may be wholly or partially transparent to electromagnetic radiation and/or electric/magnetic fields.

The at least one flow control structure may comprise a stirrer or other mechanism to create a turbulent flow of the air or other gas.

The means for providing electromagnetic radiation may comprise an energy generator and/or a source of electromagnetic radiation, optionally a microwave source.

The means for providing electromagnetic radiation may comprise or be under control of a controller configured to control the electromagnetic energy provided to the treatment region. The controller may control the power, or amount of energy, of the electromagnetic radiation delivered to the treatment region, for example to provide a desired amount or range of power or energy

The means for providing electromagnetic radiation may be configured to produce, at at least one position, optionally throughout, the treatment region and/or waveguide, and/or resonant cavity an electric field of between 50 Vm⁻¹ and 100,000 Vm⁻¹, optionally between 50 Vm⁻¹ and 300,000 Vm⁻¹, optionally between 50 Vm⁻¹ and 50,000 Vm⁻¹, optionally between 100 Vm⁻¹ and 10,000 Vm⁻¹, optionally between 200 Vm⁻¹ and 10,000 Vm⁻¹, optionally between 200 Vm⁻¹ and 5,000 Vm⁻¹.

The electromagnetic radiation may comprise microwave radiation.

The electromagnetic radiation may comprise electromagnetic radiation having a frequency or frequencies in a range 0.5 GHZ to 500 GHZ, optionally in a range 1 GHz to 500 GHz, optionally in a range 5 GHz to 100 GHz, optionally in a range 7 GHz to 10 GHz. The or an operating frequency may be in a range 1 GHz to 500 GHz, optionally in a range 5 GHz to 100 GHz, optionally in a range 7 GHz to 10 GHz.

The electromagnetic radiation may comprise continuous wave electromagnetic radiation

The electromagnetic radiation may comprises pulsed electromagnetic radiation

The pulsed electromagnetic radiation may comprise a series of pulses with a time gap between pulses in a range 0 is to 100 s optionally in a range 1 s to 60 s

The electromagnetic radiation may comprise modulated electromagnetic radiation, optionally modulated in accordance with at least one of an amplitude modulation technique a frequency modulation technique, a pulse width modulation control scheme, and/or an on/off keying (OOK) scheme.

The electromagnetic radiation may have a pulse modulation rate in a range 0.1 KHz to 100 kHZ, optionally in a range 1 kHz to 10 kHZ

The electromagnetic radiation may have a frequency modulation rate in a range 0.1 KHz to 1 MHZ, optionally in a range 1 kHZ to 100 kHZ.

The means for providing electromagnetic radiation to the treatment region may be configured to sweep or step the frequency of the electromagnetic through at least part or all of the or a frequency range

The apparatus may comprise at least one feed for introducing the electromagnetic radiation to the treatment region and/or waveguide structure, wherein optionally the at least one feed is provided at a side of the treatment region and/or waveguide structure and/or the at least one permeable structure is provided at at least one end of the treatment region and/or waveguide structure

The at least one permeable electromagnetic boundary structure may comprise reactive material and/or a reactive coating that interacts with the at least one pathogen and/or that provides or releases in response to the electromagnetic radiation a substance that interacts with the at least one pathogen, for example thereby to destroy or otherwise treat the at least one pathogen.

The apparatus may comprise or form part of at least one of an air conditioning unit or system, an air or other gas filtration unit or system, a heating apparatus or system, air flow equipment, a ventilator unit or system, a medical ventilator unit or system, a vacuum cleaner, a hand dryer, a hair dryer, a dehumidifier.

In a further aspect, which may be provided independently there is provided a method of treatment of air or other gas containing at least one pathogen or of a surface region that may include at least one pathogen, the method comprising applying microwave radiation to the air or other gas or the surface region so as to at least partially destroy the at least one pathogen and/or so as to render the at least one pathogen less harmful and/or less infectious to humans and/or to other living subjects.

The method may be performed using an apparatus as claimed or described herein

Features in one aspect may be provided as features in any other aspect as appropriate. For example, features of a method may be provided as features of an apparatus and vice versa. Any feature or features in one aspect may be provided in combination with any suitable feature or features in any other aspect.

### BRIEF DESCRIPTION OF THE DRAWINGS

Embodiments of the invention are now described, by way of non-limiting example, and are illustrated in the following figures, in which:—

FIG. 1 is a diagrammatic illustration of an electromagnetic energy transmission system to apply controlled amounts of energy to an airflow according to an embodiment;

FIG. 2 is a diagrammatic illustration of an electromagnetic energy resonant cavity system to apply controlled amounts of energy to an airflow according to an embodiment;

FIG. 3 is a diagrammatic illustration of various air permeable electromagnetic boundary arrangements to facilitate transfer of electromagnetic energy to an airflow according to an embodiment;

FIG. 4 is a diagrammatic illustration of a cylindrical waveguide electromagnetic energy exchanger to apply controlled amounts of energy to an airflow according to an embodiment;

FIG. 5 is a diagrammatic illustration of a waveguide baffle arrangement according to an embodiment;

FIGS. 6A and 6B are schematic illustrations of apparatus that provides circularly polarised electromagnetic radiation according to an embodiment,

FIG. 7 is a schematic end view of the embodiment of FIG. 6A showing, in top and bottom figures, electric field strength as a function of position at different times illustrating a rotation of the polarised mode with varying phase of applied radiation;

FIG. 8 is a schematic illustration of an apparatus that is configured to produce a circularly polarised cavity mode in a waveguide of the apparatus;

FIG. 9 is a schematic illustration of an apparatus according to an embodiment, which is configured to produce a circularly polarised propagating mode in a waveguide of the apparatus using a tuning arrangement;

FIG. 10 is a schematic illustration of an apparatus according to an embodiment, which is configured to produce a circularly polarised propagating mode in a waveguide of the apparatus using a tuning arrangement and which includes an open port at an end face of the waveguide;

FIG. 11 is a schematic illustration of an apparatus according to an embodiment, which is configured to produce a circularly polarised propagating mode in a waveguide of the apparatus using a tuning arrangement and which includes a coaxial to waveguide transitional structure at one end of the waveguide;

FIGS. 12A and 12B are schematic illustrations of pars of an apparatus according to an embodiment, which includes a waveguide and a port at an end of the waveguide that has a smaller cross-sectional area than the waveguide;

FIGS. 13A and 13B are schematic illustrations of apparatus according to an embodiment, which includes a waveguide and an air, gas or vapour supply port in a side-wall of the waveguide,

FIG. 14 is a schematic illustration of an apparatus according to an embodiment, which includes a waveguide and a port that has at least one selected dimension (e.g. diameter or width/height) less than a cut-off of the waveguide that supports an electromagnetic energy mode at a frequency of operation;

FIG. 15 is a schematic illustration of an apparatus according to an embodiment, which includes stacked waveguides;

FIG. 16 shows performance results for an apparatus including stacked waveguides according to an embodiment;

FIG. 17 is a schematic illustration of a further apparatus according to an embodiment, which includes stacked waveguides;

FIG. 18 is a schematic illustration of a further apparatus according to an embodiment, which includes stacked waveguides;

FIG. 19 is a schematic illustration of a further apparatus according to an embodiment, which includes stacked waveguides; and

FIG. 20 is a schematic illustration of a system for treating and performing measurements on air or gas containing pathogens, according to an embodiment.

### DETAILED DESCRIPTION

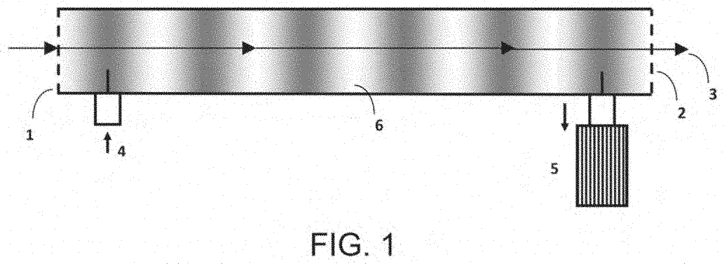

An electromagnetic energy transmission system to apply controlled amounts of energy to airflow is illustrated in FIG. 1. In this illustration there is a waveguide having an air permeable electromagnetic boundary 1 and air permeable electromagnetic boundary 2 which permits inlet and outlet airflow 3. This airflow may be forced air as part of, for example, an air conditioning or air filtration system or could be natural airflow. Within this apparatus there is a microwave input feed point 4 and an output terminating load 5 connected to another feed. The energy is delivered into the system via the feed 4 and travels as an electromagnetic transmission mode 6. This transmission mode is constrained by the air permeable electromagnetic boundaries 1 and 2. The energy in this arrangement passes via transmission along the waveguide into the output termination load 5 and is absorbed and converted to heat. In this arrangement the airflow passes through the waveguide which contains the transmitted electromagnetic mode to exchange energy into pathogens suspended in the airflow. The region within the waveguide where the electromagnetic energy interacts with the pathogens, for example to at least partially destroy and/or deactivate the pathogens and/or render the pathogens less harmful and/or less infectious to humans and/or to other living subjects, may be referred to as a treatment region.

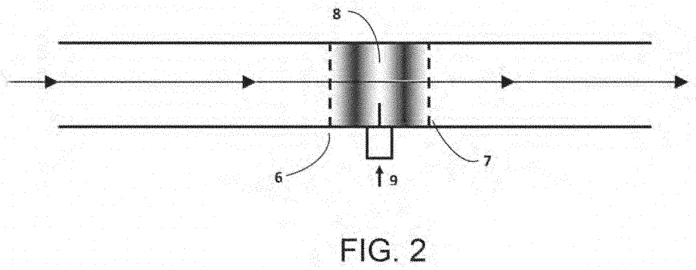

An alternative arrangement is illustrated in FIG. 2. In this illustration the air permeable electromagnetic boundaries 6 and 7 are formed within the air guide and are arranged to create a resonant electromagnetic cavity 8. In this arrangement there is a microwave input feed point 9 which delivers microwave energy into the resonant cavity where it is greatly enhanced by the resonant Q of the cavity mode. The energy in this arrangement is stored inside the cavity and the airflow passes through this zone to exchange energy into pathogens suspended in the airflow.

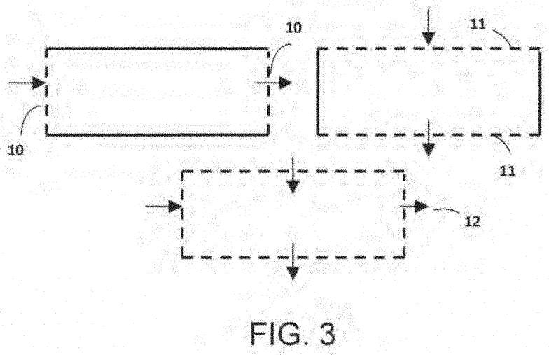

A series of alternative air permeable electromagnetic boundary arrangements are illustrated in FIG. 3. In these air permeable electromagnetic boundary end walls 10 are represented by dashed lines, air permeable electromagnetic boundary side walls 11 are represented by dashed lines and a combination of both air permeable electromagnetic boundary end walls and air permeable electromagnetic boundary side walls 12 are represented by dashed lines. Each of the illustrations in FIG. 3 are end-on views of a rectangular waveguide according to an embodiment, with solid lines indicating, non-gas permeable walls of the waveguide.

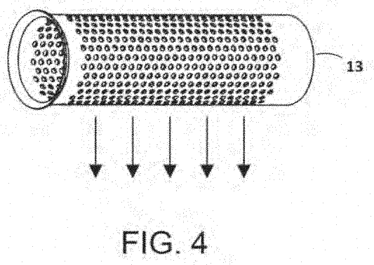

A further alternative embodiment is illustrated in FIG. 4, where a cylindrical waveguide electromagnetic energy exchanger to apply controlled amounts of energy to airflow is illustrated. In this arrangement the wall is perforated by holes (circular, square or rectangular or any other shape)

The air permeable electromagnetic boundary arrangements can, for example, be wire mesh, solid metal with machined holes, electroformed, moulded, wrapped, bonded or otherwise formed conductive elements.

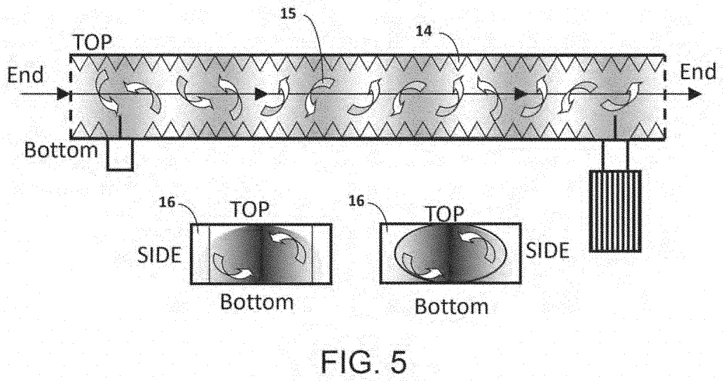

An enhancement is illustrated in FIG. 5, which shows both a side-on view of an embodiment and end-on views of variants, where a baffle arrangement 14 is introduced into the waveguide to perturb the airflow 15. In this example of a rectangular waveguide the baffle 14 is placed along the sidewall. This baffle may be formed from any low loss dielectric insulating material such as PTFE, FEP or other low loss ceramic material. This baffle could also be solid metal corrugations formed or added to the waveguide walls. In other airflow arrangements this perturbation structure could be arranged in other configurations, perturbation could also be created by secondary airflows from fans or bleed vents. The end-on views of the variants in FIG. 5 also show a flow control structure 16 included in a rectangular waveguide, and the flow control structure 16 in these variants may be a baffle or corrugated structure, it may be placed at the sides of the waveguide cavity or may have any shape to contain the airflow. If the flow control structure is a dielectric the electromagnetic energy will mainly pass through it unimpeded. The airway or gas flow path will however be constrained as the flow control structure will reduce the volume inside the waveguide. The wide arrows 15 are intended to represent turbulent airflow caused by the irregular or other perturbing flow control structure (e.g. baffle) inside the guide. In the embodiment, the airflow at the sides of the guide wall where the electric field may be the weakest is reduced and air is also circulated or at least follows a non-linear path inside the guide to maximise exposure to the peak electric field in the centre of the guide.

Any suitable flow control structures as well as instead of a baffle can be provided in other embodiments, for example at least one fin, baffle or vent and can be used to provide turbulent flow and/or mixing and/or non-laminar flow of the air or other gas, and/or to control flow of the air or other gas. In some embodiments a flow control structure comprises at least one fan or vent configured to provide a secondary flow or air or other gas in addition to the flow of air or other gas via the main input.

The at least one flow control structure in some embodiments comprises a tubing or guiding structure or other conduit that has a desired form, for example a coiled, curved or otherwise shaped structure that is configured to receive the flow of air or other gas and that passes through the treatment region. The tubing or guiding structure or other conduit may be wholly or partially transparent to electromagnetic radiation and/or electric/magnetic fields.

The at least one flow control structure in some embodiments comprises a stirrer or other mechanism to create a turbulent flow of the air or other gas.

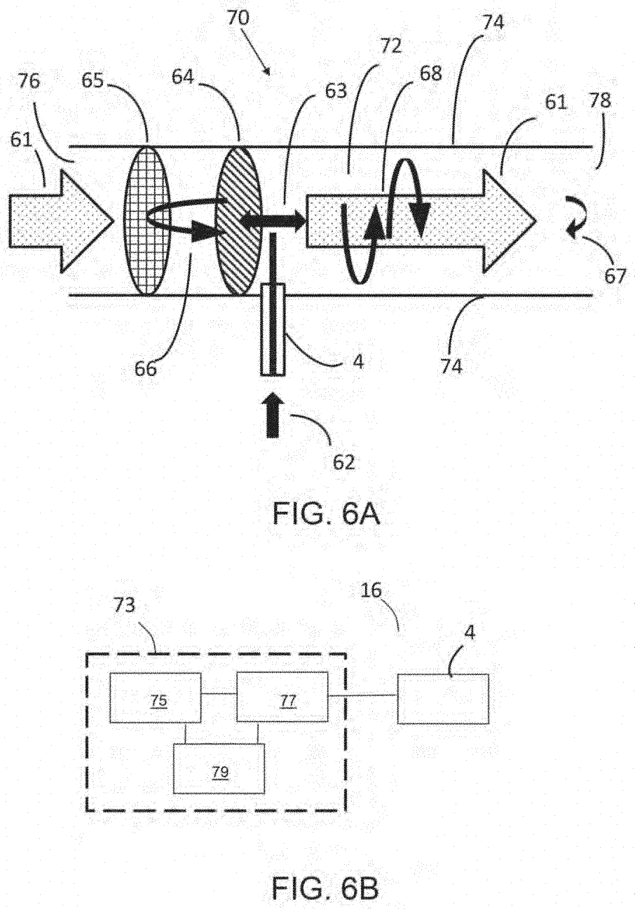

FIG. 6A shows schematically an alternative embodiment of an apparatus comprising a microwave waveguide that permits flow 61 of air or other gas, optionally a vapour or aerosol, through a permeable barrier. This waveguide has additional features to enable the creation of a circular polarised mode that may be a propagating mode or a cavity mode The apparatus uses electromagnetic radiation of circular polarisation in treating a flowing medium. Thus, the flow will pass through a rotating electromagnetic field that may more uniformly and evenly couple energy into particles or constituents of that flow

The apparatus of FIG. 6A includes a waveguide 70 comprising walls 74 forming a cavity 72 therebetween.

A feed structure 4, also referred to as a feed point, is provided that extends into the cavity 72 of the waveguide and that is used to deliver electromagnetic energy into the cavity 72 of the waveguide 70. The feed structure 4 includes a radiating element that delivers the electromagnetic energy into the cavity 72 of the waveguide 70 as electromagnetic radiation.

In the embodiment of FIG. 6A, the feed structure 4 includes a coaxial cable and the radiating element comprises a centre conductor of the coaxial cable from which outer layers have been removed and which is positioned in the waveguide to excite one or more particular electromagnetic modes. The shape, size, position and other properties of the radiating element can vary in different embodiments based, for example, on known microwave and waveguide techniques. The radiating element can have any suitable shape, position and orientation and is not limited to extending perpendicularly from a side wall 74 of the waveguide. In some embodiments, the feed structure, or at least the radiating element of the feed structure, is made as thin as possible so as not to block the airflow. In some embodiments, the feed structure includes a perturbation or guide structure (for example, a dielectric baffle or metallic fin) that is configured to direct or perturb the flow or air or other gas to help mix the gas or add vortices, for example to provide more even exposure to the electromagnetic energy and/or to provide a more homogeneous distribution of pathogens.

The feed structure 4 can also be used in any of the other illustrated embodiments, but alternatively any other suitable type of feed structure for delivering electromagnetic energy into a waveguide can be used in those illustrated embodiments or variants thereof.

The apparatus of FIG. 6A also includes a wire polariser 64 between the feed structure 4 and one end 76 of the waveguide 70. The wire polariser 64 in operation of the apparatus provides a circularly polarised mode of the electromagnetic radiation in the cavity 72. The use of a wire polariser can provide for minimal restriction of airflow due to the minimal solid area of the wire while providing a desired polarisation. Any other suitable polarising structure can be used in alternative embodiments, as well as or instead of the wire polariser In some embodiments, wire or other conductive elements of the polariser are patterned or otherwise provided on a dielectric insert having perforations, holes or slots to permit airflow.

The apparatus of FIG. 6A also includes an electromagnetic boundary structure in the form of a conductive mesh or perforated metal wall (e.g. a permeable barrier) 65 located between the wire polariser 64 and the end 76 of the waveguide. The conductive mesh or perforated metal wall 65 is permeable to air or other gases whilst, in operation, constraining the electromagnetic field in the cavity 72 of the waveguide from exiting the waveguide structure via end 76. The conductive mesh or perforated metal wall 65 also reflects a portion of the electromagnetic energy 66 received from the feed structure 4. This polarised reflected portion of energy combines with the polarised incident energy 63 to create a circular polarised electromagnetic wave 68 through which the air or other gas 61 passes in operation. The electromagnetic boundary structure may include a water-resistant or other protective outer layer, for example a plating or coating, to protect against the effects of atmospheric moisture or other substances that may be present in the air or other gas

The air or other gas 61 to be treated enters the waveguide cavity 72 through the end 76 of the waveguide 70. In the apparatus of FIG. 6A the open end 76 is can be referred to as a gas input. In alternative embodiments an input nozzle or port structure can be provided, for example at end 76 or any other suitable position, as a gas input, for example which may be connected to a conduit for transporting the gas to the input. In some embodiment, a driving device, for example a pump or fan, may be provided connected to the gas input and/or output for driving flow of the air or other gas to and/or from the treatment region via the permeable structure(s).

In the apparatus of FIG. 6A, end 78 of the waveguide cavity 72 is open and provides an output for the air or other gas, and the electromagnetic radiation in the waveguide can also pass out of the open end 78. In variants of the apparatus of FIG. 6A, an air permeable electromagnetic boundary 2 is provided at or near end 78 and an output termination load 5 is also included, for example as in the apparatus of FIG. 1 The electromagnetic boundary at end 78 may, for example, comprise a conductive mesh or perforated metal wall and may, for example at least partially guide and/or confine the electromagnetic radiation provided by the feed structure, for example to at least partially confine the electromagnetic radiation to the treatment regions, for instance by at least partially blocking, absorbing or reflecting the electromagnetic radiation. The electromagnetic boundary of the embodiment of FIG. 6A can provide, for example. 99.99% attenuation in field strength within 10 mm or less. For example, in one mode of operation within 10 mm of the boundary the electric field strength may drop from 3,000 V/m to 0 V/m for 10 W delivered by the feed structure. This is for 10 W delivered to the feed port. Such rapid and high attenuation of field strength can be obtained using any suitable permeable structures, for example conducing meshes or nets or perforated sheets or other structures including apertures of suitable size less than wavelength(s) of radiation in the waveguide, in accordance with known electromagnetic techniques.

The region of the waveguide cavity 72 between permeable structure 65 and end 78 of the waveguide 70 can be considered to be a treatment region and is a region where the electromagnetic energy 63 provided by the electromagnetic radiation provided by the feed structure can interact with pathogens that may be present in the air or other gas 61 that passes through the apparatus. The embodiments of FIGS. 1 to 5 also include treatment regions, within the waveguides, comprising the regions where the electromagnetic energy can interact with pathogens in the flow of air or other gas. It will be understood that, depending on the embodiment, the pathogens may only be destroyed, inactivated or rendered less harmful or infectious by interaction with the electric and/or magnetic fields provided by the electromagnetic radiation at certain parts or sub-regions of the treatment region, for example by way of oscillation or acoustic resonance or vibration. In other embodiments, depending on the electric and/or magnetic fields provided by the electromagnetic radiation, such effects may be obtained substantially throughout the treatment region. The electromagnetic radiation provided by the generator or other source, for example via the feed structure, can establish desired electric and/or magnetic waves and/or fields and/or modes and/or distribution of electromagnetic energy in the waveguide structure, whether static or propagating.

In the apparatus of FIG. 6A, the waveguide is of WR112 form in accordance with the Electronic Industries Alliance (EIA) designation (or WG15 according to RSCS standard, or R84 in accordance with IEC standard). The waveguide in this embodiment has cross-sectional dimensions of 1.122 Inches (28.4988 mm) width×0.497 Inches (12.6238 mm) height, a recommended frequency band of 7.05 to 10 GHz (which will sustain the mode in frequency(ies) of interest in the present embodiment), a cutoff frequency of lowest order mode of 5.26 GHz, and a cutoff frequency of upper mode of 10.52 GHz.

In a variant of the apparatus of FIG. 6A, to enhance the air flow capabilities a larger waveguide may be used, for example a WR137 rectangular waveguide in in accordance with the EIA designation (or WG14 according to RSCS standard, or R70 according to the IEC Standard). Such a waveguide has dimensions of 1.372 Inches (34.8488 mm)×0.622 Inches (15.7988 mm) height, a recommended frequency band of 5.85 to 8.20 GHz, a cutoff frequency of lowest order mode of 4.301 GHz. and a cutoff frequency of upper mode of 8.603 GHz.

In variants of the apparatus of FIG. 6A, and various other embodiments, cylindrical waveguides may be used, for example: WC-94 (EIA designation) with inside diameter (round) of 23.83 mm, cut-off frequency for air-filled of 7.377 GHz, and recommended frequency range for TE mode of 8.49 to 11.6 GHZ, WC109 (EIA designation) with inside diameter (round) of 27.79 mm, cut-off frequency for air-filled of 6.326 GHz, and recommended frequency range for TE mode of 7.27 to 9.97 GHZ, WC128 (EIA designation) with inside diameter (round) of 32.54 mm, cut-off frequency for air-filled of 5.402 GHz, and recommended frequency range for TE mode of 8.49 to 11.6 GHZ

The waveguide 70 and waveguide walls 74 can comprises any suitable materials and structures, for example in accordance with known microwave waveguide or other electromagnetic waveguide techniques. For example in some embodiments the waveguide walls 74 are formed by forming a void in a dielectric body, for example as part of an injection moulding process, and/or by drilling, erosion, or any other suitable forming technique, and then providing the inner or outer surface of the body with a conductive layer, for example a metal layer (e.g. silver, gold, nickel or an alloy thereof) for instance by performing a suitable coating, painting and/or deposition process using any suitable known technique. In other embodiments the walls 74 may be formed solely or predominantly of metal, rather than comprising a conductive layer on or in a dielectric material. In some embodiments, the walls of the waveguide may be constructed wholly or partly from permeable structures, for example conductive mesh or perforated metal material, that may support the internal transmission of electromagnetic energy and also permit lateral or cross-flow of air or other gas. The waveguide can be formed to have any desired cross-sectional profile in embodiments, for example round, square or rectangular or other shape, and any length profile for example a constant or varying/tapering cross-sectional profile with longitudinal position if desired.

In certain embodiments, the waveguide walls 74 can be provided with apertures or conduits, for example gas input(s) and/or output(s) and may include or have attached thereto, other features for example, feed structure(s) and/or radiating element(s)

In the apparatus of FIGS. 6A and 60, a microwave generator 73 or other electromagnetic source is connected or connectable to the feed structure 4. The microwave generator 73 comprises any suitable microwave source 75, for example any suitable oscillator device operable to generate an alternating signal at a desired frequency or frequency range, 7 to 9 GHz in the present embodiment, and an amplifier 77 connected to the source 75 and operable to amplify the signal from the source to a higher power level (for example 10-200 W or any other suitable power level) The amplifier in the present embodiment comprises a solid state amplifier or travelling wave tube (TWT), although any other suitable amplifier can be used. The amplifier has either SMA or N-Type coaxial inputs or outputs, but may have any other suitable inputs and/or outputs. Any suitable oscillator can be used, for example any dielectric resonator oscillator (DRO) or any crystal oscillator (XO) provided they possess a desired frequency bandwidth

The generator 73 also includes a controller 79, which is operable to control operation of the oscillator and/or the amplifier, thereby to control one or more properties of the microwave signal or other signal that is generated. The controller 79 may control any desired properties of the electromagnetic radiation that is generated to treat particular pathogens.

In operation, the controller 79 controls the generator 73 to provide electromagnetic radiation of properties suitable for treatment of a particular pathogen or pathogens of interest. The size of the virion or other pathogen will generally determine, or at least affect, the electromagnetic frequency required for it to resonate or at least oscillate, and thus for it is be destroyed, deactivated or otherwise treated. As it becomes smaller the frequency increases. At the scale of influenza and coronavirus particles or other pathogens of similar size, for example with 60 to 140 nm, or 80 nm to 100 nm, diameters or lengths, microwave frequencies may generally be required to acoustically damage the viral envelope.

In one mode of operation of the embodiment of FIGS. 6A and 6B, the generator operates to provide power/electric field levels of at least around 200 V/m or higher at at least some positions within the waveguide/treatment region of the apparatus, at at least one frequency in a range 8.0 GHz to 8.4 GHz in order to treat the influenza A virus subtype H3N2 In the case of influenza A virus subtype H3N2 other suitable power/electric field values can for example be obtained from, for example. 7] Sun, C., Tsai, Y., Chen, Y. E. et al. Resonant Dipolar Coupling of Microwaves with Confined Acoustic Vibrations in a Rod-shaped Virus Sci Rep 7, 4611 (2017).

In a simulation for a 10 W feed input can create an electric field of level of approximately 70 V/cm (7,000 V/m) with a waveguide height of 12.68 mm in the embodiment of FIG. 6A.

For other pathogen types, suitable frequency and power ranges can be determined based, for example, on known or measured size, dipolar properties or other properties of the pathogen, and/or from known or measured resonance frequencies, and/or from modelling for example as described in 7] Sun, C., Tsai, Y., Chen, Y. E. et al. Resonant Dipolar Coupling of Microwaves with Confined Acoustic Vibrations in a Rod-shaped Virus. Sci Rep 7. 4611 (2017) Direct measurements of pathogen destruction/inactivation can also be performed following treatment using the apparatus for different frequencies, power levels and/or other parameters to determine preferred or optimal operating parameters

By using a combination of a waveguide and permeable electromagnetic boundary structures, and thus providing treatment of pathogens within the waveguide, accurate control of electric and/or magnetic fields experienced by the pathogens can potentially be provided. For example, electric field profiles that would be experienced by the pathogens in the treatment region can be determined using know waveguide modelling or measurement techniques. For example, any suitable modelling software, for example Ansys HFSS: High Frequency Electromagnetic Field Simulation Software, can be used to determine electric field profiles. Furthermore, in various particular embodiments any desired modes, including propagating modes and cavity modes can be established, and polarisation and other effects can be used, which can ensure that the pathogens may encounter a range of electric field strengths/powers including desired electric field/strengths powers. For example, it can provided in some embodiments that there is a more uniform exposure of the air or other gas to the electromagnetic energy by using polarisation effects and/or time- and/or position-varying electric fields and/or by providing physical mixing or turbulence in the air or other gas. Field strengths arising from the applied electromagnetic radiation at at least some points encountered by the airflow in the waveguide structure can potentially be significantly higher, for example ten times or more higher; than might be obtained in practice using at least some techniques based on transmission of radiation into free space. For example electric field strengths of thousands or tens of thousands of Vm⁻¹ may be provided at at least some points.

In addition in some embodiments the apparatus is attached to air conditioning or any other of a range of suitable types of equipment, to ensure that pathogens in air other gas passing through such equipment is treated by the radiation in an efficient manner in the controlled treatment region of the waveguide.

In some embodiments, sources and controllers with any suitable characteristics may be used, and the electromagnetic radiation may, for example, comprise electromagnetic radiation having a frequency or frequencies in a range 0.5 GHz to 500 GHz, for example 915 MHz or 868 MHz, optionally in a range 0.5 GHz to 100 GHz, optionally 5 GHz to 100 GHz optionally in a range 7 GHz to 10 GHz or any other suitable value The electromagnetic radiation may comprise continuous wave electromagnetic radiation or pulsed electromagnetic radiation depending on the embodiment and mode of operation. The electromagnetic radiation may for example comprise a series of pulses with a time gap between pulses in a range 0.1 s to 100 s, optionally in a range 1 s to 60 s or any other suitable value. The electromagnetic radiation may comprise modulated electromagnetic radiation, optionally modulated in accordance with at least one of an amplitude modulation technique, a frequency modulation technique, a pulse width modulation control scheme, and/or an on/off keying (OOK) scheme. The electromagnetic radiation may, for example, have a pulse modulation rate in a range 0.1 KHz to 100 kHZ, optionally in a range 1 kHz to 10 kHZ or any other suitable value. The electromagnetic radiation may, for example, have a frequency modulation rate in a range 0.1 KHz to 1 MHZ, optionally in a range 1 kHZ to 100 kHZ or any other suitable value. The electromagnetic radiation provided by the feed structure may, for example, have a power or peak power in the range 0.1 W to 100 W, 10 W to 100 W, 20 W to 50 W or any other suitable value. The peak power may be the maximum during a particular treatment duration. In some embodiments, the power density may vary across the longitudinal axis of the waveguide or treatment region, for example along a propagation direction, and be substantially uniform along the width or height, e.g. from top to bottom, in the middle of the waveguide or treatment region. Suitable values of such parameters can be selected by a user, using the controller, to treat particular pathogens of interest. It will be understood that references to electromagnetic radiation in a waveguide may refer to electromagnetic modes, for example cavity modes or propagating modes or any other suitable modes, established in the waveguide.

The controller 79 may be in any suitable form, for example a suitably programmed PC or other computer, or may comprise ASIC(s) or FPGA(s) or any suitable combination of hardware and software The controller 79 may be configured to control the generator to apply a sequence of microwave or other electromagnetic treatment programs, for example a sequence of pulses, or continuous wave radiation, with desired properties. The controller may be configured so that a user can select stored programme(s) and/or so that the user can control particular parameters as desired, for example via a suitable user interface.

Although details of each of the oscillator, amplifier, and controller used in the embodiment of FIGS. 6A and 6B are provided above, any suitable components can be used, and embodiments are not limited to the particular components described in relation to FIGS. 6A and 6B.

The waveguide 70, waveguide walls 74, cavity 72, feed structure 4, polariser 64, electromagnetic boundary and/or generator 73 or other electromagnetic source, and/or the operating parameters of FIGS. 6A and 6B can be also be used in any of the illustrated or other embodiments, or any other suitable type of waveguide, waveguide walls, feed structure, polariser, electromagnetic boundary and/or generator or other electromagnetic source and/or operating parameters can be used in those embodiments. Size and shape of the cavity and other features may be varied between embodiments to provide desired electromagnetic properties, for example desired distribution of electric and/or magnetic field strength with position in the waveguide cavity in accordance with waveguide design and/or electromagnetic modelling techniques.

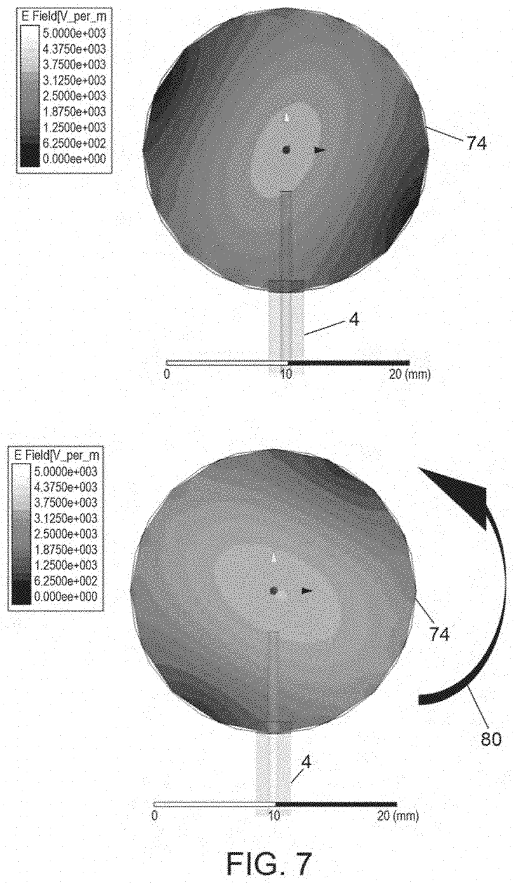

FIG. 7 is a schematic end view of a variant of the embodiment of FIG. 6 showing, in top and bottom figures, electric field strength (in Vm⁻¹) as a function of position at different times, and illustrating a rotation of the polarised mode with varying phase of radiation applied by the feed structure. The electric field strength values shown in the figure were calculated using Ansys HFSS: High Frequency Electromagnetic Field Simulation Software. The electromagnetic mode in the waveguide can be seen to rotate 80 with the phase of the incident signal The rotating of the mode can provide an effective way to ensure interaction between electromagnetic energy and pathogens in the treatment region e.g. within the cavity of the waveguide, as the electric field strength vanes with time and position, potentially reducing the chances of a pathogen passing only through low field regions.

The electric filed scale included in FIGS. 7 to 11 represents shading with values varying from 0 to 5,000 Vm⁻¹.

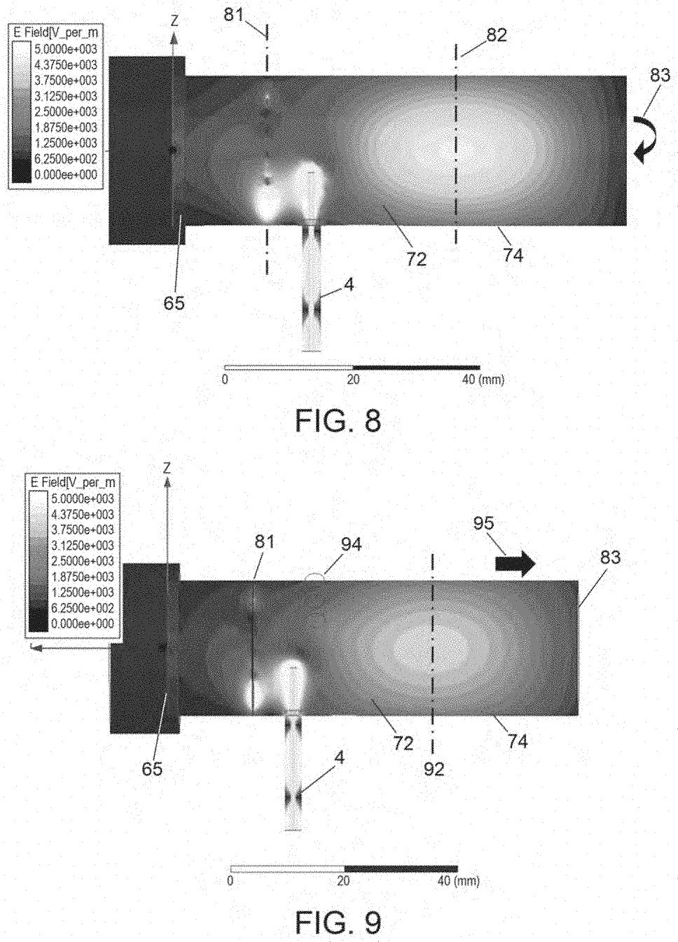

FIG. 8 shows an apparatus that is configured to provide a cavity mode, for example a circularly polarised cavity mode. This TM mode is bounded by a magnetic field boundary on the open face 83 of the waveguide which causes the electromagnetic mode to establish within the guide and to peak 82 a distance half way between the feed point, e.g. feed structure 4, and the end boundary In this case a polariser 81 is used, which may be of same or similar form to polariser 64 or may be any other suitable type of polariser. It can be seen that no energy can exit the guide via the permeable barrier on the left side of the illustration. Thus the electromagnetic radiation may be at least partially confined by the permeable barrier at that end of the waveguide. An advantage of a fixed mode can be significantly higher levels of stored electric and magnetic field as a result of the finesse or Q of the cavity mode resonance. In addition only one conductive electromagnet is boundary is required as the open end face provides the other magnetic H-wall boundary. TE modes can be provided in other embodiments. The apparatus of FIGS. 8, 9 and 10 provide a treatment region from permeable structure 65 to open end face 83.

In FIG. 9 a modification to the apparatus of FIG. 8 is shown, which includes a tuning structure, which may for example be referred to as a perturbation structure, 94 in the cavity. The tuning structure may modify the operation of the waveguide to support a propagating mode 95 that may operate within the same guide at a slightly different or same frequency depending upon the design of the tuning arrangement. The tuning structure may provide a conductive or dielectric perturbation into the field within the waveguide and may produce inductive or capacitive tuning effects depending upon the design. The tuning structure in the embodiment of FIG. 9 comprises a conductive pin or bolt, in this case a copper pin or bolt. In other embodiments a copper, brass, aluminium bolt and/or a silver coated or other metallic-coated pin or bolt is used as the tuning structure. In some other embodiments a dielectric slug or rod, for example a ceramic or sapphire slug or rod, is used.

By including a tuning structure and the electromagnetic boundary structure a closed system of a desired length with a propagating circularly polarised mode may be created. This can have advantages where the exposure time to the field is important and by increasing the waveguide length the transit time of the flow within the more uniform circularly polarised electromagnetic energy can potentially be increased.

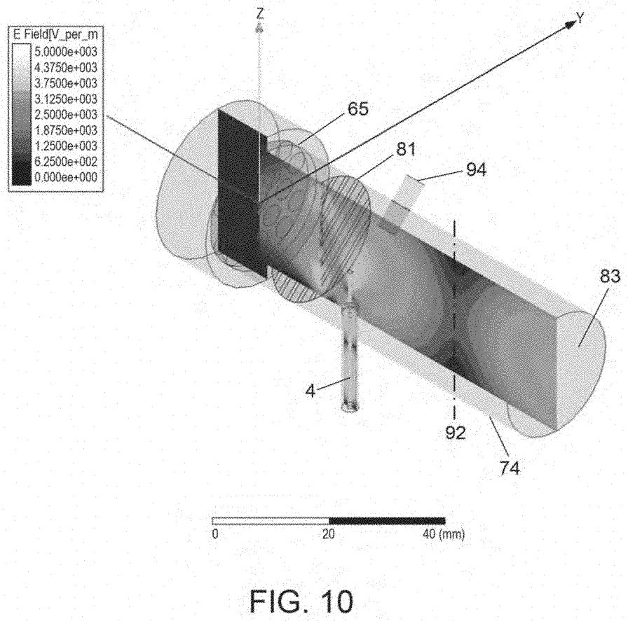

In FIG. 10 this can be seen for a static representation of the propagating wave In this case the energy has travelled out of the guide (e.g. into a boundary port at end face 83) for half of a phase cycle of the mode compared to FIG. 9. The image of FIG. 9 shows the initial propagation at 0 phase and the image of FIG. 10 shows the situation one cycle later where a null field is now in the middle 92 of the guide cavity and half the energy has exited the face 83. The port can absorb this energy in the present embodiment, but if this were a waveguide of greater length the energy would just continue to propagate along it showing multiple nulls and peaks (hot spots) in electric field.

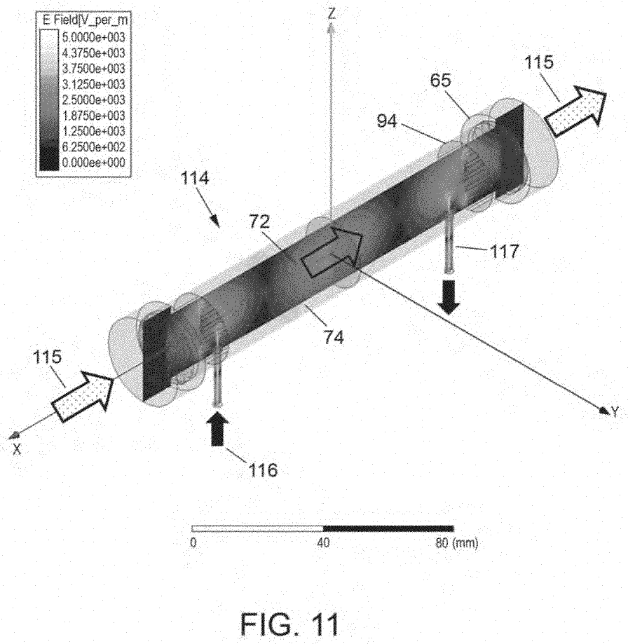

The end face open port 83 of the apparatus of FIGS. 8 and 9 can be replaced with a coaxial to and/or from waveguide transitional structure, or other structure, as illustrated in FIG. 11 where the gas flow 115 enters and exits the combined structure 114. The structure accepts electromagnetic energy 116 at one side via feed structure 4 and propagates this rotating field to a further point, for example a waveguide to coaxial cable structure 117, which may comprise or be connected to a terminating impedance or absorbing load or other absorber. Both a polariser 94 and a permeable electromagnetic boundary structure 65 may be provided at both ends of the waveguide in this apparatus. The overall length of the waveguide structure can be increased as required with the waveguide having a low propagation loss which permits an efficient way to prolong the flow/EM field interaction In this case both polarisers may be aligned, for example slanted at 45 degrees, to prevent or reduce any cancellation effect from reflected energy that may spoil the circular polarisation, for example in accordance with techniques described in 9] K J. Jeon, K J. Lee, T. K. Lee, J W. Lee and W K. Lee, Proceedings of the Fourth European Conference on Antennas and Propagation, Barcelona, Spain, 2010. pp 1-4

In some designs the perturbation may not be required if the open magnetic boundary is eliminated.

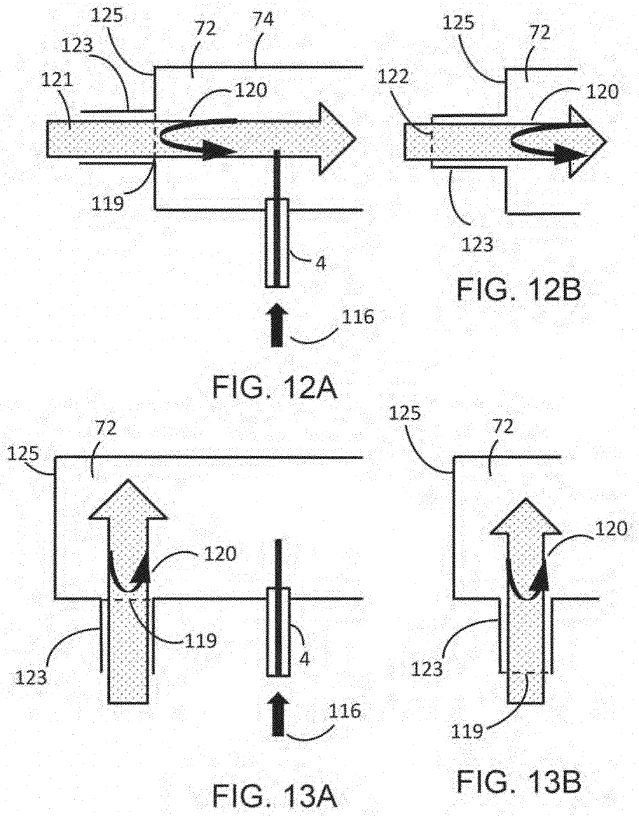

In other embodiments illustrated in FIG. 12A, an end face port structure 123 that is smaller, for example has a smaller cross-sectional area, than the guide may be used as an input to supply the air or other gas flow 121 to the electromagnetic waveguide 70 This may have a permeable electromagnetic boundary 119 at or near the inside of the guide to reflect energy 120, for example at or near an end face 125 of the cavity 72. Alternatively, the permeable electromagnetic boundary 122 could be at or near the outside of the port end face structure 123 as shown schematically in FIG. 12B. In some embodiments the end face port structure may function as a further waveguide structure FIGS. 12A and 12B only show part of the apparatus, at one end, for clarity, and it can be understood that the apparatus can include other components of the other illustrated embodiments, for example polarisers if desired, in any desired combination.

In further alternative embodiments illustrated in FIGS. 13A and 13B, a side or side face pod 123 instead of an end face port, and supplies the air or other gas flow perpendicular to the direction of the guide. Again the permeable electromagnetic boundary 119 may be provided at or near an inside surface of the waveguide, e g at or near a surface of the cavity 72, to reflect energy 120 as shown in FIG. 13A Alternatively, the permeable electromagnetic boundary 119 in this case could also be at or on near the outside of the port end face structure 123 as shown in FIG. 13B.

In another alternative embodiment the permeable electromagnetic boundary may be in the form of the input port 123 itself configured to operate as further waveguide and also functioning as in input, for example a feed port, through which the air or other gas enters. If such further waveguide that has at least one dimension 146 (e.g. diameter and/or width/height) much less than a cut-off of the waveguide that supports the electromagnetic energy mode at the frequency of operation. For example a waveguide with less than 20.24 mm diameter will not operate effectively below 8.685 GHz and a feed port diameter of 80% of the waveguide would start to attenuate the signal. A further waveguide of less than % the diameter may attenuate any second harmonic from escaping via the feed port. This below cut-off operation may suppress energy from propagating in the smaller, further waveguide (feed port) at the frequency of operation and would act like a choke or filter. Thus, an additional permeable structure 119 (e.g. a mesh, net or perforated structure) may not be needed as the blocking and/or reflection of the electromagnetic energy may be provided by the further waveguide itself even if open at both ends. The walls of the further waveguide in certain embodiments are conductive if the further waveguide is used to provide a choke effect. Alternatively or additionally, an aperture of the port or other output may be sufficiently small enough to prevent the electromagnetic energy exiting.

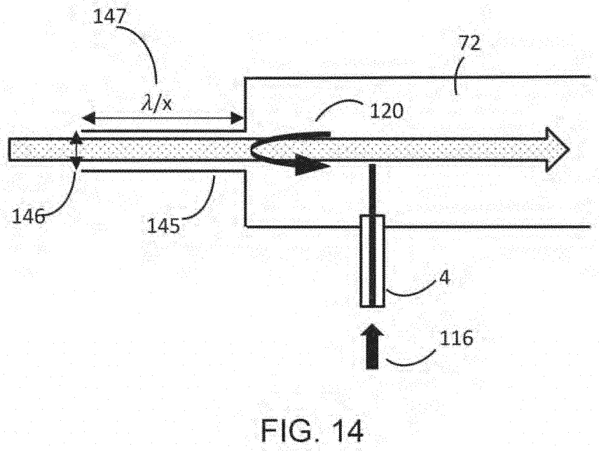

Alternatively or additionally, as illustrated in FIG. 14 a further waveguide 145, which may also operate as a feed port for the gas at the end face, may have a property of being a fractional number of wavelengths 1/n or ½\hat{ }n (where n is an integer) long 147 at the operating frequency of the waveguide 70, for example such as ½, ¼, ⅛ or any other integer number of half wavelengths. This may have the effect of transforming an open circuit to present an equivalent short circuit at the boundary and therefore suppress or further choke any leakage of electromagnetic energy. This may limit the flow into the guide but may increase pressure or produce a natural turbulence (mixing vortex) due to the transition in sizes between the inlet 123 and the guide cavity 72.

For embodiments that include a polariser, for example to provide circular polarisation, the waveguide cavity may have a cylindrical or square cross-sectional shape or have any suitable selected symmetry. A feed port, for example feed port 145 operating as further waveguide, may have a rectangular, square or cylindrical cross-section in such embodiments.

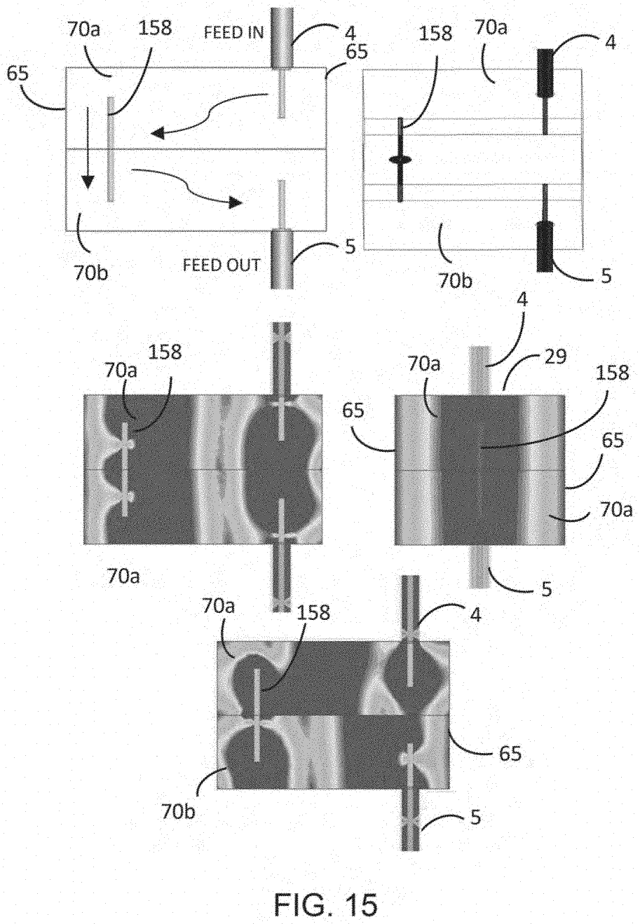

In other embodiment, for example as illustrated in FIG. 15, waveguides 70a, 70b, for example rectangular waveguides are stacked to flow air, gas, vapour through a side wall 65 of the waveguides to take advantage of the longitudinal uniformity of the electromagnetic fields within rectangular waveguides The waveguides can be stacked using a coaxial coupling 158 between each waveguide 70a, 70b such that electromagnetic energy is input via feed 4 into first waveguide 70a, passes through an electromagnetically impermeable wall between the first and second waveguides via and coaxial coupling 158 and exits the second waveguide 70b via terminating load 5 or other structure Five separate views of the embodiment, or variants thereof, are provide in FIG. 15, from different perspectives and with or without variation of calculated electric field values represented by shading

In the embodiments of FIG. 15, flow of air or other gas is through sidewalls 65 of each waveguide 70a, 70b and air or other gas passes through either one waveguide 70a or the other waveguide 70b. The stacked waveguides thus provide an increased surface area for air or other gas to pass through for treatment. In variants of the embodiments, or in other stacked waveguide embodiments, one or more conduits for air or other gas may be provided between the waveguides 70a, 70b such that air or other gas passes from one waveguide to the other before being output, thus increasing a duration of exposure to electromagnetic energy

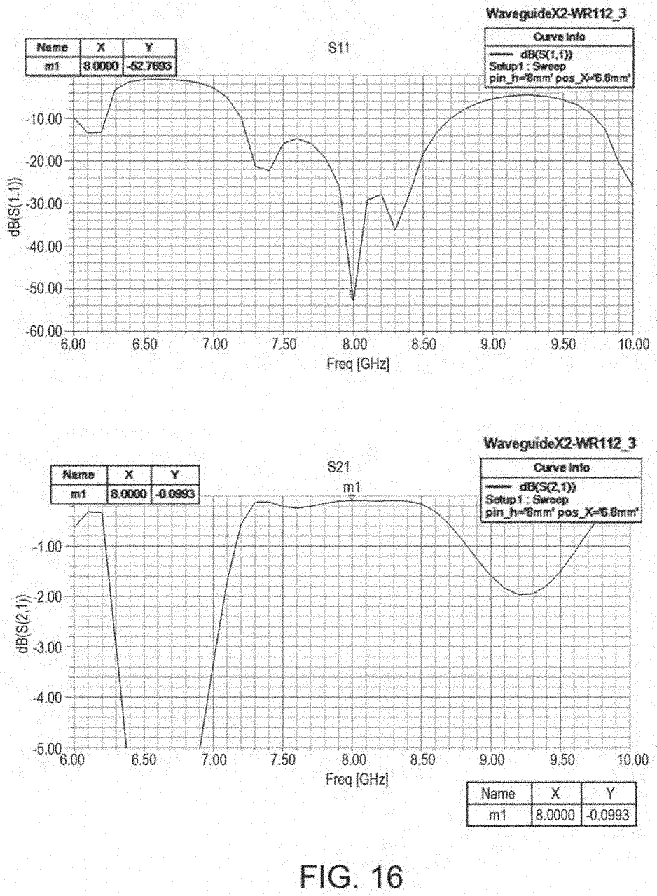

Results obtained using a pair of stacked guides are provided in FIG. 16, which shows plots of S11 and S12 parameters as a function of frequency for a pair of stacked waveguides. In this example this arrangement accepts and transfers power efficiently indicating that acceptable operation may be obtained for embodiments with larger numbers of stacked waveguides. The top graph of FIG. 16 indicates shows the frequency range for which a return loss, or S11 parameter value, is below a level of −15/−20 dB covers a useful frequency range for particular embodiments, including any suitable frequency between 7.5-8.5 GHz. The bottom graph indicates an insertion loss of −0.0993 dB which equates to 97.74% of the energy being transmitted from feed input to output.

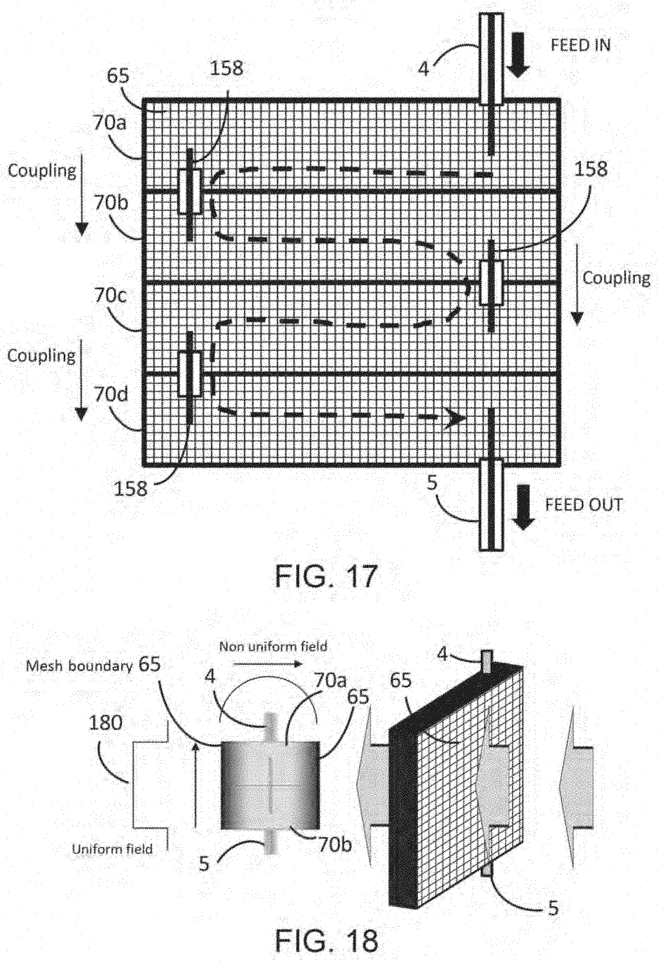

An apparatus with multiple stacked waveguides is illustrated schematically in FIG. 17 In this case a larger planar structure can be created to interface with a larger area of flow by stacking waveguides 70a, 70b, 70c, 70d as a stack of waveguides. In the case where multiple stages are desired, to enhance the performance the planar layers can themselves be stacked adjacently, for example one stack of waveguides 70a, 70b, 70c, 70d behind the other so that air or other gas flows through each stack in turn, to further increase the duration of exposure to the electromagnetic energy. Such a stacked arrangement is illustrated schematically in FIG. 19. This arrangement may also permit other, for example conventional, gas filtering methods to be used between each waveguide filter stack to enhance performance. For example, a conduit may join each pair of stacks and a gas filter may be provided in the conduit.

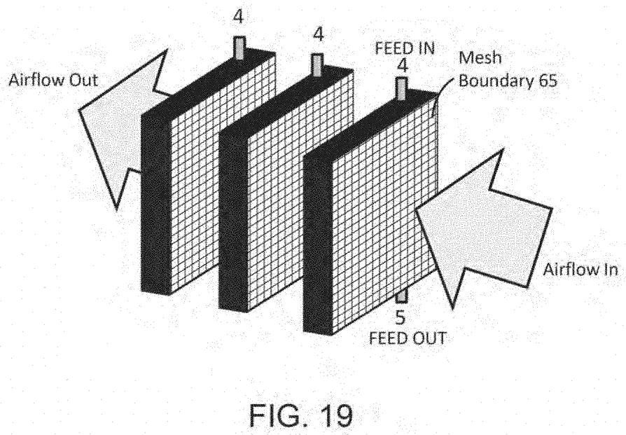

FIG. 18 shows another embodiment of a stacked arrangement in both side and perspective views, and indicating the uniformity of the electromagnetic field in a longitudinal direction of the rectangular waveguide, and the varying electromagnetic field in a perpendicular direction.

In other aspects of the invention the permeable electromagnetic boundary may also include surface coatings or combinations thereof such as an oxide or carbon material that could react or catalyse due to interaction with the microwave electromagnetic energy to release a Reactive Oxygen Species (ROS) that may further enhance the neutralising effects on any pathogens. This method may absorb some more of the energy, however in the waveguide transmission model the transmitted energy is almost entirety absorbed into the terminating load so this energy can be further utilised for this secondary means as required without significantly limiting the primary performance.

In the case where an absorptive load is used the airflow may be subsequently used to cool the microwave load component or the heat from this may be used to add heat into the airflow The systems described herein may be incorporated by design into existing air conditioning or air filtration systems or be retrofitted to existing systems. This technology may be used as a sole technology or in combination with other filtration technology such as HEPA, UV, electrostatic, vortex etc.

The apparatus according to embodiments can be used to treat a variety of pathogens, by suitable choice of electromagnetic parameters. The treatment can comprise, for example, at least partially destroying and/or deactivating and/or inactivating the at least one pathogen, and/or rendering the at least one pathogen less harmful and/or less infectious to humans and/or to other living subjects. The electromagnetic radiation provided by the apparatus may be such as to provide oscillation, optionally acoustic resonance, of the at least one pathogen if present in air or other gas in the treatment region, and the oscillation and/or acoustic resonance may be such as to at least partially destroy and/or alter physical structure of and/or at least partially deactivate the at least one pathogen.

A variety of pathogens may be treated, according to embodiments For example, the at least one pathogen can comprise at least one virus particle, the at least one pathogen can comprise one or more viral respiratory pathogens, the at least one pathogen can comprise at least one virus particle of the Family Orthomyxoviridae and/or Coronaviridae particles, the at least one pathogen can comprise at least one virus particle of the Genra Influenzavirus (i.e. Influenza or ‘flu’) and/or Coronavirus, the at least one pathogen can comprise at least one influenza virus particle, the at least one pathogen can comprise at least one virus particle classified as any of an Influenza virus A, Influenza virus B, Influenza virus C or Influenza virus D particle, the at least one pathogen can comprise at least one virus particle classified as any of an Avian ‘flu’ (A/H5N1 subtype), a Canine ‘flu’ (Influenza virus), an Equine ‘flu’ (Influenza virus) or a Swine ‘flu’ (A/H1N1 subtype) particle, the at least one pathogen can comprise at least one virus particle of the Genera Coronavirus, the at least one pathogen can comprise at least one Coronavirus particle, the at least one pathogen can comprise at least one virus particle classified as belonging to any of the following Genera: Alpha-, Beta-. Gamma-, and Deltacoronavirus, the at least one pathogen comprises at least one virus particle classified as any of the following:
(i) the SARS Coronavirus; or
(ii) the MERS Coronavirus; or
(iii) SARS-CoV-2 (aka COVID-19).

The at least one pathogen may have a non-spherical structure and/or have a non-spherical distribution of electrical charge

The generator 73 and controller 79, or other source of electromagnetic radiation can be used to provide electromagnetic radiation to treat any particular pathogen of interest, for example based on expected acoustic resonances or oscillation frequencies of the pathogen of interest and/or based on routine experimentation to determine the parameters of electromagnetic radiation that are effective to treat the pathogen of interest. In some embodiments, sweeping or stepping of frequency and other parameters can be performed to ensure that the pathogens experience electromagnetic energy with properties suitable to destroy or otherwise treat the pathogen.

Apparatus according to embodiments is applicable to, for example, air conditioning or air filtration systems or air flow equipment in buildings such as offices, clean rooms, hotel rooms, schools, hospitals, supermarkets, universities, churches, stadiums, oil rigs or in transport applications such as trains, aeroplanes, ships, cars, buses, lorries or in commercial applications such as toilet hand dryers, dehumidifiers, portable air conditioners, portable air filters, vacuum cleaners. In such systems or equipment, a gas input and/or output of apparatus according to any suitable embodiments can be connected to an input; output, or internal conduit of such equipment or systems to treat air or other gas, optionally aerosols, passing into and/or out of such equipment or systems.

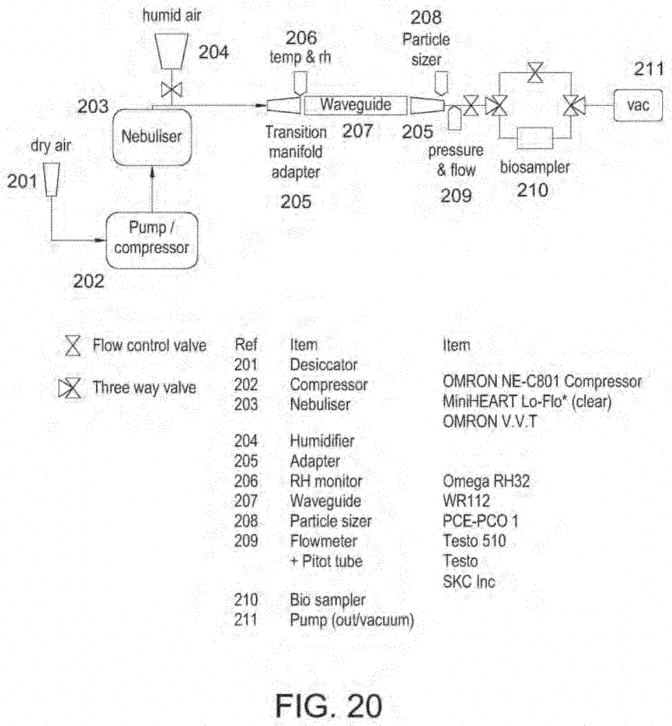

FIG. 20 is a schematic illustration of system for treating and performing measurements on air or gas containing pathogens, the system including an apparatus as described according to illustrated embodiments including a waveguide structure 207 and any associated components, which may be in the form of waveguide structure 70 as described. An associated generator and controller as described in relation to other embodiments are also included The system can be used for example, to treat air or other gas containing pathogens, measure the effectiveness of the treatment using biosampler 210. Thus, effectiveness of operating parameters, for example electromagnetic parameters, in respect of, for example, particular pathogens, flow rates and humidity conditions, may be tested, and operating parameters selected.

The system includes a desiccator 201, a compressor 202 (for example an OMRON NE-C801 compressor), a nebuliser 203 (for example an MiniHEART Lo-Flo® (clear)), a humidifier 204, and adapter 205, RH monitor 206 (for example an Omega RH32), waveguide 207, particle sizer 208 (for example a PCE-PCO 1), a flow meter and Pitot tube 209 (for example a Testo 510), a biosampler 210 (for example an SKC Inc. biosampler) and a pump 211.

It will be understood that the present invention has been described above purely by way of example, and that modifications of detail can be made within the scope of the invention

Each feature disclosed in the description and (where appropriate) the claims and drawings may be provided independently or in any appropriate combination.

### REFERENCES

[1] Ford, L (2003). Estimate of vibrational frequencies of spherical virus particles. Physical review. E, Statistical, nonlinear, and soft matter physics. 67. 051924. 10.1103/PhysRevE.67.051924.
[2] S C Yang, et al. (2015) Efficient structure resonance energy transfer from microwaves to confined acoustic vibrations in viruses. Scientific reports 5, 18030
3] T Noda et al (2006) Architecture of ribonucleoprotein complexes in influenza A virus particles. Nature 439, 490-492 (2006).
4] Schoeman, D., Fielding. B. C Coronavirus envelope protein: current knowledge. Virol J 16, 69 (2019).
5] Chen N et al. (2020) Epidemiological and clinical characteristics of 99 cases of 2019 novel coronavirus pneumonia in Wuhan, China: a descriptive study The Lancet. Articles 395(10223), 507-513.
6] T M Liu et al. (2009) Microwave resonant absorption of viruses through dipolar coupling with confined acoustic vibrations. Applied Physics Letters, 94(4), 043902.
7] Sun, C., Tsai. Y., Chen, Y. E. et al. Resonant Dipolar Coupling of Microwaves with Confined Acoustic Vibrations in a Rod-shaped Virus. Sci Rep 7. 4611 (2017)
8] P Tsai and S R Malkan U.S. Pat. No. 6,428,610
9] K J Jeon. K J Lee T K Lee, J W Lee and W K Lee. “Circular polarization generating coaxial to waveguide adapter for horn antenna.” Proceedings of the Fourth European Conference on Antennas and Propagation, Barcelona Spain. 2010. pp 1-4.

1. An apparatus for electromagnetic treatment of at least one pathogen in air or other gas, comprising:
a waveguide structure including a treatment region within the waveguide structure, wherein the treatment region is configured to receive the air or other gas that contains the at least one pathogen;
at least one of an energy generator or a source of electromagnetic radiation, configured to provide electromagnetic radiation to the treatment region;
at least one structure permeable to the air or other gas and that is configured to at least partially confine the electromagnetic radiation, wherein the at least one permeable structure forms at least one boundary of the treatment region.

2. The apparatus according to claim 1, wherein the treatment region comprises at least part of a cavity in the waveguide structure.

3. The apparatus according to claim 1, wherein the at least one permeable structure is arranged to at least one of form the waveguide structure and/or to provide at least one wall of the waveguide structure.

4. The apparatus according to claim 1, wherein at least one of:
the waveguide structure and at least one of the energy generator or the source of electromagnetic radiation are configured to provide cavity mode radiation in the treatment region;
the waveguide structure and at least one of the energy generator or the source of electromagnetic radiation are configured to provide propagating electromagnetic radiation in the treatment region;
or
the waveguide structure and at least one of the energy generator or the source of electromagnetic radiation are configured to provide a desired electromagnetic mode in the treatment region.

5. The apparatus according to claim 1, further comprising at least one absorber configured to absorb the electromagnetic radiation.

6. The apparatus according to claim 1, wherein the at least one permeable structure comprises at least one of:
a metal structure;
at least one of a mesh structure, a perforated sheet, netting, or a filter;
a further waveguide.

7. The apparatus according to claim 1, wherein at least one of:
the treatment of the at least one pathogen comprises at least partially at least one of destroying or deactivating the at least one pathogen;
the treatment of the at least one pathogen comprises at least partially inactivating the at least one pathogen;
or
the treatment of the at least one pathogen comprises rendering the at least one pathogen at least one of less harmful or less infectious to at least one of humans or to other living subjects.

8. The apparatus according to claim 1, wherein the electromagnetic radiation is such as to provide oscillation, of the at least one pathogen if present in air or gas in the treatment region.

9. The apparatus according to claim 8, wherein at least one of the oscillation or the acoustic resonance is such as to at least partially at least one of destroy, alter physical structure of or deactivate the at least one pathogen.

10. The apparatus according to claim 1, wherein the at least one pathogen comprise at least one virus particle.

11. The apparatus according to claim 1, wherein at least one of:
a) the at least one pathogen comprise one or more viral respiratory pathogens;
b) the at least one pathogen comprise at least one virus particle of at least one of the Family Orthomyxoviridae or the Family Coronaviridae particles;
c) the at least one pathogen comprises at least one virus particle of at least one of the Genra Influenzavirus (i.e. Influenza or ‘flu’) or Coronavirus;
d) the at least one pathogen comprise at least one influenza virus particle.
e) the at least one pathogen comprise at least one virus particle classified as any of an Influenza virus A, Influenza virus B, Influenza virus C or Influenza virus D particle;
f) the at least one pathogen comprise at least one virus particle classified as any of an Avian ‘flu’ (A/H5N1 subtype), a Canine ‘flu’ (Influenza virus), an Equine ‘flu’ (Influenza virus) or a Swine ‘flu’ (A/H1N1 subtype) particle;
g) the at least one pathogen comprises at least one virus particle of the Genera Coronavirus;
h) the at least one pathogen comprise at least one Coronavirus particle;
i) the at least one pathogen comprise at least one virus particle classified as belonging to any of the following Genera: Alpha-, Beta-, Gamma-, and Deltacoronavirus; or
j) the at least one pathogen comprises at least one virus particle classified as any of the following:
(i) the SARS Coronavirus; or
(ii) the MERS Coronavirus; or
(iii) SARS-CoV-2 (aka COVID-19).

12. The apparatus according to claim 1, wherein the at least one pathogen has at least one of a non-spherical structure or a non-spherical distribution of electrical charge.

13. The apparatus according to claim 1, further comprising at least one polariser.

14. The apparatus according to claim 13, wherein the at least one polariser comprises at least one of at least one circular polariser or the electromagnetic radiation is circularly polarised.

15. The apparatus according to claim 13, wherein the polariser(s) and waveguide structure are configured such that the electromagnetic radiation provides at least one of an electric field or magnetic field that rotates over time with respect to a longitudinal axis of the waveguide structure.

16. The apparatus according to claim 13, wherein the at least one polariser comprises at first polariser and a second polariser each towards a respective end of at least one of the treatment region or waveguide structure and the first polariser has a desired alignment with respect to the second polariser.

17. The apparatus according to claim 1, further comprising at least one tuning or perturbation structure that is configured to affect properties of electromagnetic radiation in a cavity of the waveguide structure.

18. The apparatus according to claim 17, wherein the at least one tuning or perturbation structure is configured to provide a propagating electromagnetic mode at a selected frequency in the waveguide structure.

19. The apparatus according to claim 1, comprising a plurality of waveguide structures each including a respective treatment region, wherein at least one of the energy generator or the source of electromagnetic radiation is configured to provide electromagnetic radiation to each of the treatment regions, and the apparatus further comprises at least one conduit for the air or other gas to pass between the waveguide structures.

20. The apparatus according to claim 19, wherein at least one of the plurality of waveguide structures are in a stacked arrangement or the plurality of waveguide structures comprise a plurality of rectangular waveguides.

21. The apparatus according to claim 1, further comprising at least one of at least one input arranged to provide the air or other gas to the treatment region, or at least one output arranged for passage of the air or other gas from the treatment region.

22. The apparatus according to claim 21, wherein at least one of the at least one input is arranged to provide the air or gas through the at least one permeable structure to the treatment region or the at least one output is arranged so that the air or other gas passes from the treatment region through the at least one permeable structure to the at least one output.

23. The apparatus according to claim 21, wherein the waveguide structure comprises a waveguide including at least part of the treatment region, and the at least one input comprises a further waveguide that functions as the, or one of the, permeable electromagnetic boundary structures.

24. The apparatus according to claim 23, wherein at least one of the further waveguide has at least one of a diameter, width or height that is less than an operating or cut-off wavelength of the waveguide, or the further waveguide is configured to provide at least one of an electromagnetic choke or filter effect.

25. The apparatus according to claim 21, wherein at least one of the input or the output comprises at least one conduit, and the, or at least one of the, permeable electromagnetic boundary structures is provided in, or at an end of, said at least one conduit.

26. The apparatus according to claim 1, further comprising means for driving flow of the air or gas at least one of to or from the treatment region.

27. The apparatus according to claim 26, wherein at least one of the driving means is arranged to drive the air or gas through the at least one permeable structure to the treatment region;
or
the driving means comprises at least one of a pump or fan.

28. The apparatus according to claim 1, further comprising at least one flow control structure configured to control flow of the air or other gas.

29. The apparatus according to claim 28, wherein at least one of:
the at least one flow control structure is configured to provide at least one of turbulent flow, mixing or non-laminar flow of the air or other gas;
the at least one flow control structure comprises at least one of a fin or baffle;
or
wherein the at least one flow control structure comprises at least one fan or vent.

30. The apparatus according to claim 1, wherein the electromagnetic radiation comprises microwave radiation.

31. The apparatus according to claim 1, comprising at least one feed for introducing the electromagnetic radiation to at least one of the treatment region or waveguide structure, wherein at least one of the at least one feed is provided at a side of at least one of the treatment region or waveguide structure or the at least one permeable structure is provided at at least one end of at least one of the treatment region or waveguide structure.

32. The apparatus according to claim 1, wherein the at least one permeable electromagnetic boundary structure comprises at least one of reactive material or a reactive coating that at least one of interacts with the at least one pathogen or that provides or releases in response to the electromagnetic radiation a substance that interacts with the at least one pathogen thereby to destroy or otherwise treat the at least one pathogen.

33. The apparatus according to claim 1, wherein the apparatus comprises or forms part of at least one of an air conditioning unit or system, an air or other gas filtration unit or system, a heating apparatus or system, air flow equipment, a ventilator unit or system, a medical ventilator unit or system, a vacuum cleaner, a hand dryer, a hair dryer, a dehumidifier.

34. A method of treatment of air or other gas containing at least one pathogen, the method comprising applying microwave radiation to the air or other gas at least one of so as to at least partially destroy the at least one pathogen or so as to render the at least one pathogen at least one of less harmful or less infectious to at least one of humans or to other living subjects.

35. An apparatus for electromagnetic treatment of at least one pathogen in air or other gas, comprising:
a waveguide structure including a treatment region within the waveguide structure, wherein the treatment region is configured to receive the air or other gas that contains the at least one pathogen;
means for providing electromagnetic radiation to the treatment region;
at least one structure permeable to the air or other gas and that is configured to at least partially confine the electromagnetic radiation, wherein the at least one permeable structure forms at least one boundary of the treatment region.
